(19) United States
(12) Patent Application Publication
(10) Pub. No.: US 2025/0312099 A1
(43) Pub. Date: Oct. 9, 2025

(54) PHOTOTHERAPY APPARATUS

(71) Applicant: TECURE, INC., Nam-gu Busan (KR)

(72) Inventor: Vangia TRUONG, Busan (KR)

(21) Appl. No.: 18/866,395

(22) PCT Filed: Sep. 14, 2023
(86) PCT No.: PCT/KR2023/013843

§ 371 Date: Nov. 15, 2024

(30) Foreign Application Priority Data
Sep. 16, 2022 (KR) 10-2022-0117256

(51) Int. Cl. A61B 18/24; A61B 17/00; A61B 18/00; A61B 18/20; A61B 18/22; A61B 90/00

(52) U.S. Cl. CPC A61B 18/24; A61B 2017/00398; A61B 2018/00023; A61B 2018/00244; A61B 2018/00488; A61B 2018/00577; A61B 2018/00589; A61B 2018/00714; A61B 2018/00744; A61B 2018/00773; A61B 2018/00791; A61B 2018/00875; A61B 2018/206; A61B 2018/2238; A61B 2090/3966

(57) ABSTRACT

A phototherapy apparatus includes a light generator configured to generate a laser beam; an optical fiber coupled to the light generator and including a light transfer portion configured to transfer the laser beam and a light scattering portion configured to output the laser beam in a radial direction; a first tube into which the optical fiber is inserted, and configured to inject fluid into one end; a second tube into which the first tube is inserted, and configured to discharge the fluid in a region with an outer surface of the first tube; a guide portion configured to accommodate the optical fiber by forward movement of the optical fiber and disposed on the same axis as the first tube; and a balloon configured to surround one ends of the first tube and the second tube and the guide portion and to expand and reduce according to injection and discharge of the fluid.

### TECHNICAL FIELD

The present disclosure relates to a phototherapy apparatus using a balloon, and particularly, a phototherapy apparatus which may perform phototherapy without damaging surrounding tissues using a balloon.

### BACKGROUND ART

Gastroesophageal reflux disease (GERD) is a common digestive disorder with an estimated prevalence of 18-25% in North America and has increased steadily over the past several decades. GERD may occur when stomach acid refluxes into the esophagus due to a weak or relaxed lower esophageal sphincter (LES). The LES is a junction between the esophagus and the stomach, known as the muscular ring, and is formed as a smooth muscle layer and maintains tonic contraction through muscle and nerve factors.

Generally, patients with GERD may experience major symptoms such as chest pain, heartburn discomfort, dysphagia, and dysphagia. In the case in which GERD is left untreated, complications of GERD may include esophageal ulcers, esophageal strictures, and esophageal erosion. A variety of medical and surgical treatments have been developed to treat GERD directly or indirectly.

Although endoscopic therapy has evolved as a potentially safe and effective treatment option for GERD, in the FDA-approved GERD market, the Medigus ultrasonic surgical endostapler (MUSE™, Medigus, Omer, Israel), transoral incisionless fundus surgery (EsophyX, EndoGastric Solutions, WA, USA), and Stretta therapy (Restech, Houston, TX, USA) have been used. Medigus ultrasonic surgical endoscopic surgery is a transoral incisionless anterior fundus augmentation using an ultrasound-integrated surgical stapler under endoscopic guidance. Stretta therapy remodels the sphincter by contacting directly multiple electrodes in the muscularis propria of the LES and applying RF energy thereto. The diameter of the LES may be reduced to reduce the frequency of stomach contents refluxing into the esophagus. However, these procedures are often unknown and are associated with a lack of normalization of gastric acid exposure in many patients, and limited efficacy in healing.

Also, since these procedures require advanced technology and a relatively long surgery, the risk of complications such as dysphagia, chest pain, sore throat, bleeding, and perforation may increase.

Thus, additional improvements may still be required to secure the effectiveness, costs, and treatment safety of endoscopic treatment devices for treating gastroesophageal reflux disease.

### DETAILED DESCRIPTION OF PRESENT DISCLOSURE

### Technical Problems to Solve

The present disclosure is provided to address the issues above, and the purpose of the present disclosure is to provide a phototherapy apparatus which may perform phototherapy without damaging surrounding tissues using a balloon.

However, the technical purpose which the embodiment seeks to accomplish is not limited to the technical purposes described above, and other technical purposes may be included.

### Solution to Problem

According to an embodiment of the present disclosure, a phototherapy apparatus includes a light generator configured to generate a laser beam; an optical fiber coupled to the light generator and including a light transfer portion configured to transfer the laser beam and a light scattering portion configured to output the laser beam in a radial direction; a first tube into which the optical fiber is inserted, and configured to inject fluid into one end; a second tube into which the first tube is inserted, and configured to discharge the fluid in a region with an outer surface of the first tube; a guide portion configured to accommodate the optical fiber by forward movement of the optical fiber and disposed on the same axis as the first tube; and a balloon configured to surround one ends of the first tube and the second tube and the guide portion and to expand and reduce according to injection and discharge of the fluid.

In an embodiment, the phototherapy apparatus may further include a guide rail disposed between the first tube and the guide portion, and configured to guide movement of the optical fiber from the first tube to the guide portion.

In an embodiment, the guide portion has a circular, elliptical, streamlined, polygonal, or irregular cross-sectional shape and has a diameter decreasing in a direction away from the first tube.

In an embodiment, one end of the first tube is disposed between one end of the second tube and the guide portion.

In an embodiment, the first tube and the guide portion are spaced apart from each other, such that the fluid is discharged between an outer surface of the first tube and the second tube after the fluid is injected into the balloon from one end of the first tube.

In an embodiment, the phototherapy apparatus may further include a fluid adjustment portion configured to control a flow rate and a temperature of the fluid.

In an embodiment, the phototherapy apparatus may further include an optical fiber moving portion configured to control the optical fiber f to perform translational movement in an axial direction of the optical fiber or rotational movement in a circumferential direction of the optical fiber; and a motor connected to the other end of the optical fiber.

In an embodiment, the optical fiber moving portion is configured to move the optical fiber in a first mode in which the light scattering portion is positioned in the second tube, and in a second mode in which the light scattering portion is positioned between the second tube and the guide portion.

In an embodiment, a length in an axial direction of the light scattering portion is smaller than a length in an axial direction of the balloon.

In an embodiment, at least one of the first tube or the balloon is transparent such that the laser beam passes therethrough.

In an embodiment, the phototherapy apparatus may further include a sensor array disposed on a surface of the balloon and configured to measure at least one of temperature, tissue deformation, pH, and mucosal impedance.

In an embodiment, the phototherapy apparatus may further include at least one of a first radio marker disposed on one side of the light scattering portion or a second radio marker disposed on one side of the balloon.

In an embodiment, the phototherapy apparatus may further include a tip portion disposed on a front end of the balloon; and a guide wire configured to guide movement of the balloon, wherein the guide wire is configured to penetrate the tip portion, to extend along one side of the balloon, and to be inserted into one end of the second tube.

According to an embodiment of the present disclosure, a phototherapy apparatus includes a light generator configured to generate a laser beam; an optical fiber coupled to the light generator and including a light transfer portion configured to transfer the laser beam and a light scattering portion configured to output the laser beam in a radial direction; a first tube into which the optical fiber is inserted, and configured to inject fluid; a second tube into which the first tube is inserted, and configured to discharge the fluid in a region with an outer surface of the first tube; a third tube connected to one end of the first tube and configured to accommodate the optical fiber by forward movement of the optical fiber; and a balloon configured to surround one ends of the first tube and the second tube and the third tube and to expand and reduce according to injection and discharge of the fluid, wherein the third tube includes a hole to inject the fluid into the balloon.

In an embodiment, the third tube is transparent such that the laser beam passes therethrough.

In an embodiment, the fluid is injected into the balloon through the hole in the third tube and is discharged between an outer surface of the first tube and the second tube.

In an embodiment, the first tube and the third tube are configured as an integrated tube.

In an embodiment, a hole in the third tube is positioned closer to one side opposite to the first tube among both sides of the balloon.

In an embodiment, on a plan view, the hole has a circular, elliptical, streamlined, slit, polygonal, or irregular shape.

In an embodiment, the hole includes a plurality of holes disposed in a circumferential direction or an axial direction of the third tube.

### Advantageous Effects of Invention

According to an aspect of the present disclosure, the phototherapy apparatus may perform phototherapy without damaging surrounding tissues using a balloon.

### BRIEF DESCRIPTION OF DRAWINGS

FIG. 1 is a diagram schematically illustrating overall components of a phototherapy apparatus according to an embodiment of the present disclosure.

FIGS. 2a and 2b are diagrams schematically illustrating a portion of optical fiber and a balloon portion of a phototherapy apparatus according to an embodiment of the present disclosure.

FIG. 3 is a diagram schematically illustrating an optical fiber moving portion of a phototherapy apparatus according to an embodiment of the present disclosure.

FIGS. 4A and 4B are diagrams schematically illustrating positions of optical fiber during treatment of a phototherapy apparatus according to an embodiment of the present disclosure.

FIG. 5 is a diagram schematically illustrating an example in which a phototherapy apparatus according to an embodiment of the present disclosure is inserted into a human body for treatment.

FIG. 6 is a diagram schematically illustrating an optical fiber moving portion and an inlet and an outlet for fluid of a phototherapy apparatus according to an embodiment of the present disclosure.

FIG. 7 is a diagram schematically illustrating movement of fluid in an inlet and an outlet for fluid of a phototherapy apparatus according to an embodiment of the present disclosure.

FIG. 8 is a diagram schematically illustrating movement of fluid in a balloon of a phototherapy apparatus according to an embodiment of the present disclosure.

FIG. 9 is a diagram schematically illustrating the state of tissue according to presence or absence of a cooling process after phototherapy of a phototherapy apparatus according to an embodiment of the present disclosure.

FIGS. 10A and 10B are diagrams schematically illustrating a portion of optical fiber and a balloon portion of a phototherapy apparatus according to the other embodiment of the present disclosure.

FIG. 11 is a diagram schematically illustrating movement of fluid in a balloon of a phototherapy apparatus according to an embodiment of the present disclosure.

FIG. 12 is a diagram schematically illustrating a hole of a third tube of a phototherapy apparatus according to an embodiment of the present disclosure.

### BEST MODE FOR INVENTION

The advantages and features of the present disclosure, and the method for implementing the same will become apparent with reference to the embodiments described below in detail with the attached drawings. However, the present disclosure is not limited to the embodiments disclosed below and may be implemented in various different forms, and the embodiments are provided to allow the disclosure of the present disclosure complete and to fully inform a person skilled in the art to which the present disclosure belongs of the scope of the invention, and the present disclosure is defined only by the scope of the claims. Accordingly, in some embodiments, well-known process steps, well-known device structures, and well-known techniques are not specifically described to avoid ambiguity in interpretation of the present disclosure.

The same reference numerals refer to the same elements throughout the specification.

The terminology used herein is for the purpose of describing embodiments and is not intended to limit the present disclosure. In the specification, a singular term includes a plural form unless otherwise indicated. The terms “comprises” and/or “comprising” used herein do not exclude the presence or addition of one or more other components, steps, operations, and/or elements mentioned therein. The terms “connected” or “coupled” used herein may indicate electrical connection or electrical connection (e.g., coupling) of the mentioned components but are not limited thereto, and do not exclude the presence or addition of one or more other components, steps, operations, and/or elements expected by a person skilled in the art to which the present disclosure belongs.

Unless otherwise indicated, the entirety of terms (including technical and scientific terms) used in this specification may be commonly understood by those skilled in the art to which the present disclosure belongs. Also, terms defined in commonly used dictionaries will not be ideally or excessively interpreted unless explicitly specifically defined.

The phototherapy apparatus according to an embodiment of the present disclosure may be inserted into the oral cavity and may be positioned on the sphincter of the esophagus to treat gastroesophageal reflux disease, or the like. However, an embodiment thereof is not limited thereto, and the phototherapy apparatus according to the present disclosure may be used for various purposes such as treating urinary incontinence, fecal incontinence, or the like. Further, the phototherapy apparatus according to the present disclosure may be applied to various fields to implement purpose and effect by irradiating light, and is not limited to the treatment purpose.

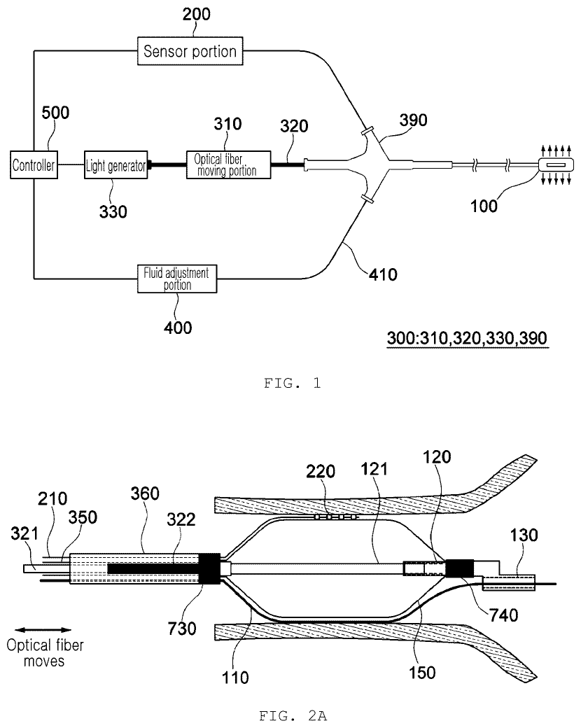

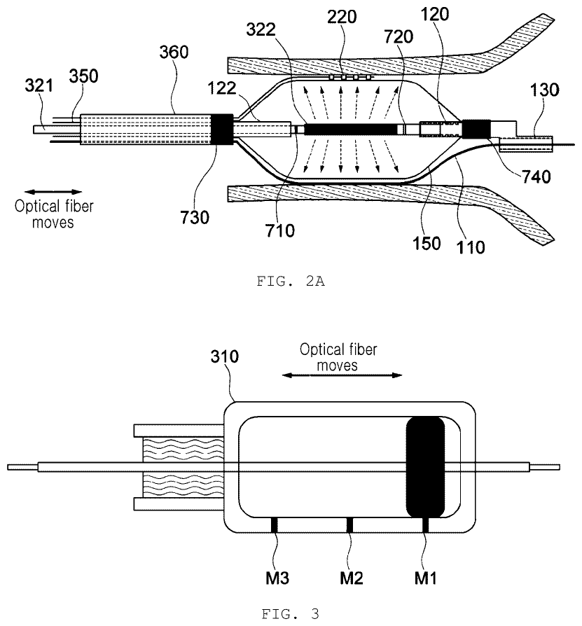

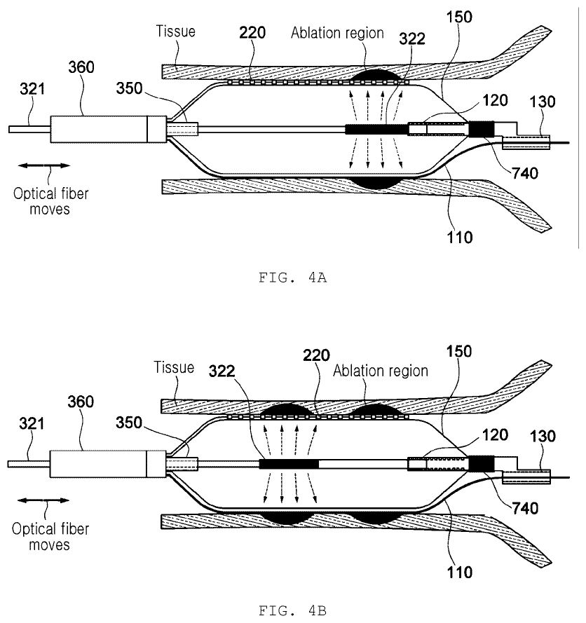

FIG. 1 is a diagram schematically illustrating overall components of a phototherapy apparatus according to an embodiment of the present disclosure. FIGS. 2a and 2b are diagrams schematically illustrating a portion of optical fiber and a balloon portion of a phototherapy apparatus according to an embodiment of the present disclosure. FIG. 3 is a diagram schematically illustrating an optical fiber moving portion of a phototherapy apparatus according to an embodiment of the present disclosure.

As illustrated in FIG. 1, a phototherapy apparatus according to an embodiment of the present disclosure may include a balloon portion 100, a sensor portion 200, a light irradiation portion 300, a fluid adjustment portion 400, and a controller 500.

In an embodiment of the present disclosure, the light irradiation portion 300 may include an optical fiber moving portion 310, an optical fiber 320, and a light generator 330. For example, the optical fiber 320 may move under control of the optical fiber moving portion 310, and the balloon portion 100 provided on a front end of the optical fiber moving portion 310 may be guided to a desired position and may be inserted, and accordingly, a light beam generated by the light generator 330 may be irradiated around the balloon portion 100. A more detailed description of each component may be provided later.

Referring to FIGS. 2a and 2b, in an embodiment of the present disclosure, the balloon portion 100 may include a guide wire 110, a guide portion 120, a tip portion 130, and a balloon 150.

In an embodiment, the guide wire 110 may penetrate the tip portion 130 and may guide the phototherapy apparatus in an axial direction of the tubular sphincter tissue. The guide wire 110 may be configured to secure an entry path of an endoscope or a narrow channel and may be positioned internally or externally of the second tube 360. That is, the guide wire 110 may be preferentially secure an insertion path of the tissue positioned in the internal region of the tube through an endoscope or a narrow channel, and thereafter, the guide wire 110 may allow the balloon portion or the endoscope to be inserted into a narrow channel along the secured insertion path, and may allow the light scattering portion 322 of the optical fiber 320 to be positioned at the treatment site.

A material of the guide wire 110 may be nitinol alloy or stainless steel, a diameter may be about 0.001-0.1 inch, and a length may be 10-1,000 cm.

In an embodiment, the guide portion 120 may be disposed on the same axis as the first tube 350 and/or the second tube 360 to accommodate the optical fiber 320 by forward movement of the optical fiber 320. For example, the guide portion 120 may be disposed on a front end (e.g., front end) of the balloon 150. In this specification, the front end (or front end) may refer to the front side with respect to the direction in which the phototherapy apparatus is inserted into the human body, and the rear end may refer to the opposite direction.

In an embodiment, as the optical fiber 320 performs translational movement, the guide portion 120 may accommodate the optical fiber 320, and may guide the optical fiber 320 to perform translational movement in an axial direction of the optical fiber 320 and/or rotational movement in the circumferential direction of the optical fiber 320. For example, the guide portion 120 may allow the optical fiber 320 inserted into the internal region to efficiently perform translational movement and rotational movement in the balloon 150, but is not limited to the above shape. For example, the guide portion 120 may have a tube shape into which the optical fiber 320 may be inserted, and a cross-sectional shape thereof in a direction orthogonal to the axis thereof may correspond to the cross-sectional shape of the optical fiber 320, but an embodiment thereof is not limited thereto. For example, the guide portion 120 may have a circular, elliptical, streamlined, polygonal, or irregular cross-sectional shape. In an example, although not illustrated, the guide portion 120 may include a groove to guide the direction of movement of the optical fiber 320 and to reduce friction on an inner surface, or may be formed smoothly. For example, the guide portion 120 may include a stopper (not illustrated) on one end such that the guide rail 121 disposed in the internal region or the optical fiber 320 may not be easily separated from the guide portion 120. In an example, although not illustrated, the guide portion 120 may be formed such that an inner diameter (e.g., inner diameter) of one end directed in the first tube 350 or the second tube 360 may be greater than that of the other portion such that the optical fiber 320 may advance from the first tube 350 and may be easily inserted into the guide portion 120. For example, the guide portion 120 may have a tapered shape having a diameter decreasing in a direction away from the first tube 350.

In an embodiment, the guide rail 121 may be disposed on one side of the guide portion 120. For example, the guide rail 121 may be extended and connected from the first tube 350 to the guide portion 120, and may guide translational movement of the optical fiber 320 between the first tube 350 and the guide portion 120. The guide rail 121 may allow the optical fiber 320 to stably perform translational movement and rotational movement without deviating from the orbit, and is not limited to the above shape. For example, the guide rail 121 may have various shapes such as a bar shape, a plate shape, a flat plate shape, a curved shape, or the like, and is not limited to any particular example as long as the guide rail 121 may at least partially support the optical fiber 320. For example, the guide rail 121 may have a shape corresponding to the shape of a portion of the optical fiber 320. For example, when the optical fiber 320 has a circular cross-sectional shape, the guide rail 121 may have a shape in contact with or surrounding a portion of an outer surface of the optical fiber 320. The guide rail 121 may include a groove guiding the direction of movement of the optical fiber 320 and reducing friction on the surface in contact with the optical fiber 320, or may be formed smoothly.

In an example, the guide rail 121 may be formed of a transparent material. For example, the guide rail 121 may be formed transparently to allow the light beam from the light scattering portion 322 to pass through. In an example, the guide rail 121 may include at least one of Pebax, polyurethane, silicone, rubber, or PEEK (polyetheretherketone), but an embodiment thereof of the present disclosure is not limited thereto. In some embodiments, the guide rail 121 may be formed opaquely depending on the intended use and wavelength conditions of the phototherapy apparatus.

In FIG. 2a, one guide rail 121 may be disposed, but an embodiment thereof is not limited thereto, and a plurality of the guide rails 121 may be formed. For example, one or more guide rails 121 may be disposed to surround an inner surface or an outer surface of the first tube 350, and in this case, the plurality of guide rails 121 may be disposed symmetrically (e.g., point-symmetrically) with respect to the axis of the first tube 350.

When the optical fiber 320 performs translational movement and rotational movement in the balloon 150, the guide rail 121 may be in contact with the optical fiber 320, or may not be in contact with the optical fiber 320, and may be in contact with the optical fiber 320 only when the optical fiber 320 deviates from the orbit, thereby preventing the optical fiber 320 from deviating. The guide rail 121 may be in contact with and fixed to the inner surface of the first tube 350 and the guide portion 120, but an embodiment thereof is not limited thereto. For example, the guide rail 121 may be coupled to one end portion of the guide portion 120 in a rail form, such that the length exposed between the first tube 350 and the guide portion 120 may be adjusted. In this case, a groove or a protrusion may be formed on the inner surface of the guide portion 120, and the guide rail 121 may include a protrusion or a groove coupled to the groove or the protrusion of the guide portion 120 on a surface directed to the inner surface of the guide portion 120, and may include a stopper fixing the position. In an example, the guide rail 121 may move or rotate together with the optical fiber 320 when the optical fiber 320 performs translational movement and rotational movement.

In an embodiment of the disclosure, an auxiliary tube 122 may be further formed to assist the optical fiber 320 to move stably from the first tube 350 to the internal region of the guide portion 120. For example, the auxiliary tube 122 may be formed of a transparent material. In an example, the auxiliary tube 122 may include at least one of Pebax, polyurethane, silicone, rubber, or PEEK (polyetheretherketone), but an embodiment thereof of the present disclosure is not limited thereto. In FIG. 2B, one auxiliary tube 122 may be connected to the first tube 350, but an embodiment thereof is not limited thereto, and the auxiliary tube 122 may be spaced apart from the first tube 350 and a plurality of the auxiliary tubes 122 may be formed between the first tube 350 and the guide portion 120.

In an embodiment, the tip portion 130 may be formed on a front end of the balloon portion 100 to guide and be inserted into the sphincter tissue. That is, when the tip portion 130 is inserted into a narrow tube, a wound or a hole may be formed on the surface of the tissue, and to reduce this, the tip portion 130 may be formed on the tip. 130 may be formed of one of Pebax, The tip portion polyurethane, silicone, rubber, or PEEK (polyetheretherketone), and may be formed with a desired size and configuration depending on the size of the balloon and the size of the tubular tissue. The tip portion 130 may include an upper tip portion into which the guide portion 120 is inserted and fixed, and a lower tip portion into which the guide wire penetrates, and the upper tip portion and the lower tip portion may be formed to have a step difference.

In an embodiment, the balloon portion 100 may be guided and inserted into, for example, sphincter tissue of the esophagus by the guide wire 110 and the tip portion 130, and the balloon 150 may be inflated by a fluid (e.g., a cooling medium including a gas such as air or a liquid) to expand the sphincter tissue.

In an embodiment, the balloon 150 may be coupled to one end of the second tube 360 to uniformly expand the stenosis or the narrowed internal structure of tissue. For example, the balloon 150 may be disposed to surround one end of the first tube 350, one end of the second tube 360, and the guide portion 120 such that the balloon 150 may expand and reduce according to injection and discharge of fluid through the first tube 350 and the second tube 360. For example, one end of the balloon 150 may be inserted into between the outer surface of the first tube 350 and the inner surface of the second tube 360, and the other end of the balloon 150 may be inserted into and fixed between the outer surface of the guide portion 120 and the inner surface of the tip portion 130.

For example, when the balloon 150 is inflated, the optical fiber 320 positioned in the internal region of the balloon 150 may not be in direct contact with the tissue during light irradiation, and the optical fiber may be positioned in the center of the tissue during treatment. In this case, the optical fiber may be positioned in the center of the tissue such that the tube tissue may be treated evenly.

In an embodiment, the balloon 150 of the balloon portion 100 may be formed of a transparent material such that the light beam uniformly distributed and emitted from the light scattering portion 322 may be irradiated externally, for example, to a sphincter tissue. For example, the material forming the balloon 150 may include acrylic, polyethylene terephthalate (PET), silicone, polyurethane, and polycarbonate, but an embodiment thereof is not limited thereto, and a general material may be selected to optimize light transmittance of the selected laser beam according to a specific wavelength, balloon shape, and target tissue.

In an embodiment, the balloon 150 may be inflated by supplying air or fluid (water, deuterium, contrast medium, or the like.) into the balloon 150. In this case, air or fluid (water, deuterium, contrast medium, or the like.) supplied into the balloon 150 may be selected to reduce absorption or scattering of the transferred laser beam wavelength. A diameter of the inflatable balloon portion 100 for tissue expansion may be 0.1-100 mm, and a length may be 1-1000 mm. The inflated balloon may have a square, circular, elliptical, streamlined, conical, tapered, or stepped shape depending on the shape of the stenotic tissue, but an embodiment thereof is not limited thereto.

Referring to FIGS. 1, 2a and 2b, in an embodiment of

the present disclosure, the light irradiation portion 300 may include an optical fiber moving portion 310, an optical fiber 320, a light generator 330, a first tube 350, and a second tube 360.

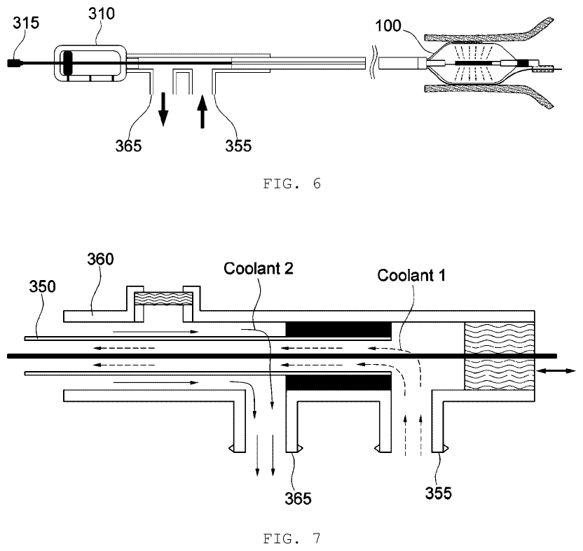

In an embodiment, the optical fiber 320 may perform translational movement in the axial direction of the optical fiber 320 and rotational movement in the circumferential direction of the optical fiber 320 under control of the optical fiber moving portion 310. To this end, a motor (315, see FIG. 6) may be disposed on a rear end of the optical fiber 320.

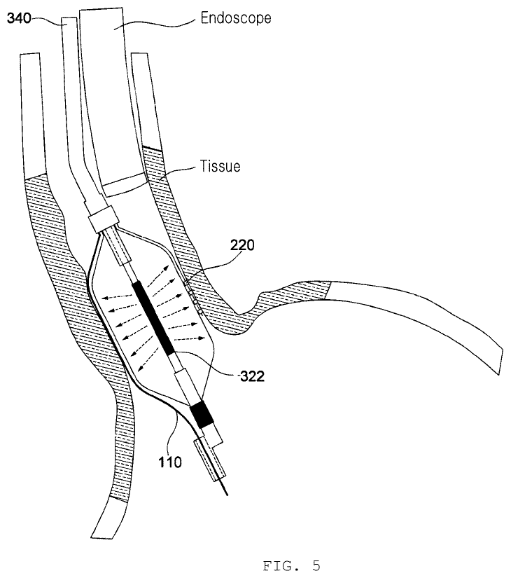

Referring to FIGS. 2a, 2b, 3, 4a and 4b, the optical fiber moving portion 310 may control movement of the optical fiber 320 in a plurality of modes. For example, the optical fiber moving portion 310 may move the optical fiber 320 in a first mode M1 (see FIG. 2a) such that the light scattering portion 322 of the optical fiber 320 may be positioned in the second tube 360, and in a second mode M2 and M3 (see FIGS. 2b, 4a, and 4b) such that the light scattering portion 322 may be positioned between the second tube 360 and the guide portion 120. For example, the first mode M1 may be an initial position of the optical fiber 320, which may be a safety position which prevents the light beam generated by the light generator 330 from being transferred to tissue. For example, the second mode M2 and M3 may include a 2-1 mode M2 in which the light beam may be irradiated in the first position in the balloon 150 and a 2-2 mode M3 in which the light beam may be irradiated in the second position in the balloon 150. For example, referring to FIG. 4a, the optical fiber moving portion 310 may move the optical fiber 320 in the 2-1 mode M2 such that the light scattering portion 322 may irradiate a light beam in the first position on the front side (e.g., distal) in the balloon 150. For example, referring to FIG. 4b, the optical fiber moving portion 310 may move the optical fiber 320 in the 2-2 mode M3 such that the light scattering portion 322 may irradiate a light beam in a second position on the rear side (e.g., proximal) in the balloon 150. In this case, the first tube 350 may be disposed to be fixed, but an embodiment thereof is not limited thereto, and the first tube 350 may move along forward movement or rotational movement of the optical fiber 320.

However, an embodiment thereof is not limited thereto, and the optical fiber moving portion 310 may move the optical fiber 320 in any number of modes, which may be three or more or less, and may include an operating bar which may be manually operable to appropriately adjust the position of the optical fiber 320. Also, although not illustrated, the optical fiber moving portion 310 may further include a switch configured to allow the optical fiber to perform rotational movement.

For example, referring to FIGS. 4a and 4b, a length in an axial direction of the light scattering portion 322 may be less than a length in the axial direction of the balloon 150. Accordingly, the light scattering portion 322 may irradiate a light beam externally in a plurality of positions in the balloon 150.

In an embodiment, the optical fiber 320 may include a light transfer portion 321 and a light scattering portion 322. The optical fiber 320 may be connected to the light generator 330, may receive a light beam (e.g., laser beam, infrared beam, or the like.) emitted from the light generator 330 through the light transfer portion 321 and may radiate the light beam through the light scattering portion 322.

The optical fiber 320 may include a core, cladding, buffer, jacket, or the like, and a diameter of the optical fiber core may be 0.01-10 mm depending on the transferred energy density, the overall diameter of the optical fiber may be 0.01-50 mm depending on an inner diameter of the endoscope, and the overall length of the optical fiber may be 0.1-10 m depending on the length of the endoscope. The length of the light scattering portion 322 in which light beam is irradiated may be 0.1-300 mm depending on the purpose of use.

The light scattering portion 322 may be formed to surround the optical fiber inserted into the balloon portion 100, and may uniformly distribute light emitted from the optical fiber 320 to the sphincter tissue. For example, the light scattering portion 322 may be formed to diffuse or scatter the light beam by embossing the entire surface or the surface portion according to a predetermined angle in the axial direction of the optical fiber. For example, the light scattering portion 322 may be formed in a cone shape. The light scattering portion 322 may emit light in the radial direction of the optical fiber 320. For example, the light scattering portion 322 may emit a light beam radially from more than 0° to 360° or less. For example, the light scattering portion 322 may be formed transparently. However, the light scattering portion 322 of the present disclosure is not limited thereto. For example, the light scattering portion 322 may be configured in the form of a coil wound on the surface of the optical fiber, and is not limited to any particular type as long as the light scattering portion 322 may irradiate a light beam in the axial direction and/or radial direction of the optical fiber 320.

A light source of the light generator 330 may be a laser beam which may simultaneously or selectively combine multiple light sources such as visible light or infrared light, and a wide range of wavelengths from 70 nm to 7000 nm in a continuous mode or a pulse model may be applied depending on the treatment purpose. The laser beam may be coupled to the optical fiber and may be irradiated, and may increase the treatment effect using a single wavelength and also multiple wavelengths.

More specifically, the light generator 330 may select a wavelength according to a heat penetration depth of a tissue target layer. That is, for heat treatment of a shallow layer of tissue, using applicable wavelengths including, for example, 405, 490, 532, 585, 755, 980, 1470, 1550, and 2200 nm, ablation, removal, destruction, and/or coagulation thicknesses of 0.1-20 mm may be generated. The radiation exposure range may be 0.01 J/cm 2-10 KJ/cm² and the power range may be 0.1 W-1000 W.

Also, examples of wavelengths applicable for heat treatment of deep tissue layers may include 630, 808, 980, 1064, and 1300 nm, and a coagulation thickness of 0.1-100 mm may be formed. In this case, the radiation exposure range may be 0.001 J/cm²-10 J/cm², and the power range may be 10 mW-100 W.

Also, by coupling two or more wavelengths, the effect of a single heat treatment may be maximized or the effect of heat treatment may be obtained simultaneously (both shallow and deep layers simultaneously), and the combination may control the spatial extent of ablation, removal, destruction, and/or coagulation in the treated tissue.

In an embodiment, the light irradiation portion 300 may include a first tube 350 into which an optical fiber 320 is inserted, and configured to insert fluid into the balloon 150 through one end thereof; and a second tube 360 into which the first tube 350 is inserted into and configured to discharge fluid in a region with the outer surface of the first tube 350. That is, the outer diameter of the first tube 350 may be smaller than the inner diameter of the second tube 360, and the inner diameter of the first tube 350 may be greater than the outer diameter of the optical fiber 320.

In an embodiment, the first tube 350 may have a form of a tube into which the optical fiber 320 may be inserted, and a cross-sectional shape in a direction perpendicular to the axis thereof may correspond to the cross-sectional shape of the optical fiber 320, but an embodiment thereof is not limited thereto. For example, the guide portion 120 may have a circular, elliptical, streamlined, polygonal, or irregular cross-sectional shape. In an example, the first tube 350 may be formed of a transparent material. For example, the first tube 350 may include at least one of Pebax, polyurethane, silicone, rubber, or PEEK (polyetheretherketone). However, an embodiment thereof is not limited thereto, and the first tube 350 may be formed in various shapes and materials depending on the intended use, durability, strength, light conditions, or the like.

The second tube 360 may be formed such that the first tube 350, the sensor wire 210, and the guide wire 110, or the like may be inserted into the internal region thereof. For example, the second tube 360 may include a plurality of lumens (not illustrated) which may accommodate the first tube 350, the sensor wire 210, and the guide wire 110, or the like. The second tube 360 may be formed of an opaque material such that a light beam from the light scattering portion 322 is not transferred externally when the light scattering portion 322 is present in the second tube 360. The second tube 360 may be formed of a generally used material in the art, and the type and shape is not limited to any particular example.

The flow of fluid through the fluid adjustment portion 400 and the first tube 350 and the second tube 360 may be described later.

A phototherapy apparatus according to an embodiment of the present disclosure may further include a sensor portion 200. For example, the sensor portion 200 may monitor physical parameters of sphincter tissue and/or the surrounding environment sensed by a sensor array 220 provided on an external surface or in an internal region of the balloon portion 100.

More specifically, a sensor array 220 may be attached to a portion of the balloon portion 100, for example, a surface, and may collect and monitor physical parameters such as a sensed temperature, tissue stress-strain, pH level, and impedance of the mucosal surface. Here, the sensor portion 200 may include monitoring parameters such as electrical signals of the nervous system through single or multiple sensors and may collect parameters through various sensors, but an embodiment thereof is not limited thereto. Also, the sensor portion 200 may transmit the sensed signals collected by monitoring to the controller 500, and the controller 500 may adjust the intensity, time, position, air injection, fluid injection, air trap, or the like of the corresponding light irradiation, such that the treatment may be performed safely during the treatment.

Referring to FIG. 1, a stop cock 390 may be used as a connection member which may integrate the optical fiber 320, the sensor wire 210 of the sensor portion 200, and the fluid supply channel (not illustrated) of the fluid adjustment portion 400 into a single tube and may insert the integrated tube into the first tube 350 and the second tube 360.

Also, according to an embodiment of the present disclosure, a radio marker may be formed to recognize a position of the light scattering portion 322 and to adjust a position in which a light beam is irradiated. For example, the phototherapy apparatus may further include first radio markers 710 and 720 disposed on at least one side of both sides of the light scattering portion 322 and second radio markers 730 and 740 disposed on at least one side of both sides of the balloon 150. Accordingly, when being inserted into the human body, the first radio markers 710 and 720 and the second radio markers 730 and 740 may be confirmed by X-ray, and the position of the light scattering portion 322 may be adjusted by moving the optical fiber 320 using the optical fiber moving portion 310.

FIG. 5 is a diagram schematically illustrating an example in which a phototherapy apparatus according to an embodiment of the present disclosure is inserted into a human body for treatment. For example, FIG. 5 is a diagram illustrating an example of applying a sphincter phototherapy apparatus using a balloon portion of the present disclosure to an esophageal sphincter.

As illustrated in FIG. 5, the lower esophageal sphincter (LES) of the esophagus may be weak or relaxed such that the LES may be closed incompletely, which may cause gastric acid to reflux into the esophagus. This may be related to additional complications of GERD, such as esophageal ulcer, esophageal stricture, and esophageal erosion. Depending on the size of the target area, a diameter of the balloon may be applied in various manners from 1 mm to 50 mm, and a general length of the catheter may be in the range of 10-500 mm, but an embodiment thereof is not limited thereto. Here, when the apparatus is inserted into the lower esophageal sphincter (LES) under an endoscope along the guide wire, and the apparatus is positioned in the LES under endoscopic visualization, the balloon may be inflated and the laser beam may be irradiated to the mucosal surface. The esophageal mucosa may be treated repeatedly several times depending on the treatment period and may be performed under endoscopic guidance. After the treatment, the balloon may be reduced and the apparatus may be removed by pulling the apparatus back along the esophagus.

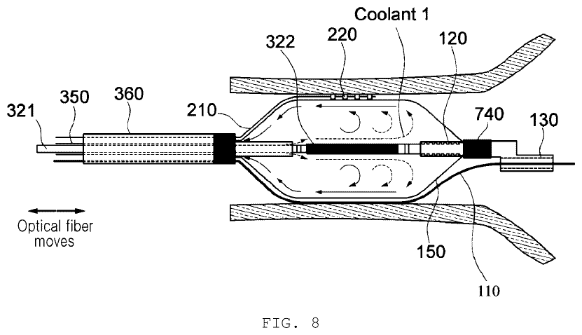

FIG. 6 is a diagram schematically illustrating an optical fiber moving portion and an inlet and an outlet for fluid of a phototherapy apparatus according to an embodiment of the present disclosure. FIG. 7 is a diagram schematically illustrating movement of fluid in an inlet and an outlet for fluid of a phototherapy apparatus according to an embodiment of the present disclosure. FIG. 8 is a diagram schematically illustrating movement of fluid in a balloon of a phototherapy apparatus according to an embodiment of the present disclosure.

Referring to FIGS. 6, 7, and 8, a phototherapy apparatus according to an embodiment of the present disclosure may further include an inlet 355 for injecting fluid into a first tube 350 and an outlet 365 for discharging fluid from a second tube 360. For example, the inlet 355 and the outlet 365 may be connected to a fluid adjustment portion 400 and a pump (not illustrated), and may allow fluid flowing in by the pump to move.

Referring to FIGS. 6, 7, and 8, in an embodiment, fluid (coolant 1) may be injected into the first tube 350 through the inlet 355. For example, fluid (coolant 1) may flow along the first tube 350 and may be injected into the balloon 150 through one end of the first tube 350. For example, one end of the first tube 350 may be spaced apart from the guide portion 120. For example, one end of the first tube 350 may protrude further into the balloon 150 than one end of the second tube 360. However, an embodiment thereof is not limited thereto, and one ends of the first tube 350 and the second tube 360 may be disposed in the same axial position.

In an embodiment, the fluid (coolant 3) injected into the balloon 150 from one end of the first tube 350 may move along the arrow and may be discharged between the outer surface of the first tube 350 and the inner surface of the second tube 360. In an embodiment, one end of the first tube 350 may be disposed between one end of the second tube 360 and the guide portion 120. The fluid (coolant 2) discharged into the second tube 360 may move along the second tube 360 and may be discharged externally through the outlet 365.

In this case, the fluid may lower or maintain the temperature in the first tube 350, the second tube 360, and the balloon 150, thereby preventing damages to human body tissue and the phototherapy apparatus, and preventing the failure of the phototherapy apparatus due to high heat generated by light beam irradiation.

In an embodiment, the fluid adjustment portion 400 may supply fluid to the balloon 150 such that the balloon 150 may inflate or reduce. In this case, the flow and temperature of the fluid in the balloon portion 100 may be controlled to an appropriate temperature and flow in the controller by data sensed by the sensor portion 200. Here, the fluid may include liquids such as distilled water, saline solution, deuterium, and contrast agents, gases such as air, and various cooling media, and may be supplied to the balloon 150 through an inflator or a pump.

However, an embodiment thereof is not limited thereto, and a plurality of the inlets and outlets may be formed, and as long as fluid may be injected and flow in the first tube 350, the second tube 360, and the balloon 150, the injection and discharge method is not limited to any particular example.

Also, in the present specification, the first tube 350 and the second tube 360 may be partially exposed in FIGS. 2a, 2b, 4a, 4b, and 8 for ease of description, but the other ends of the first tube 350 and the second tube 360 may be connected to the inlet 355 and the outlet 365, respectively, and may allow fluid to flow.

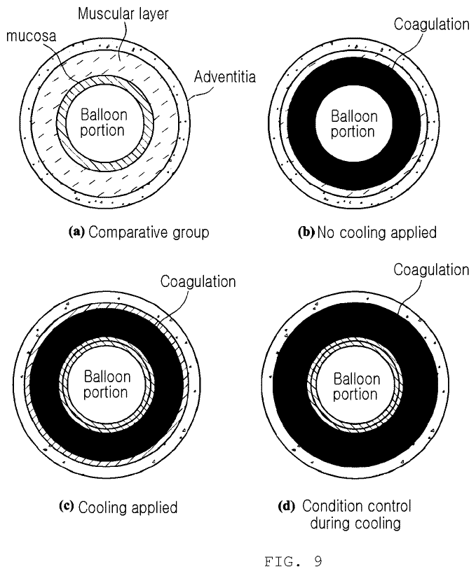

FIG. 9 is a diagram schematically illustrating the state of tissue according to presence or absence of a cooling process after phototherapy of a phototherapy apparatus according to an embodiment of the present disclosure.

FIG. 9(a) is an example of tissue into which balloon portion 100 is inserted.

Referring to FIG. 9(b), when no fluid is used in the phototherapy apparatus, thermal deformation of the internal region of tissue may occur deeply. For example, after phototherapy, the entire mucosa and muscle layer may be extensively deformed. For example, the tissue may be coagulated due to phototherapy.

Referring to FIG. 9(c), when the fluid is used in the phototherapy apparatus, thermal deformation may be reduced. For example, due to the cooling effect of the fluid, deformation of the mucosa adjacent to the balloon portion 100 may be reduced, and only the muscle layer may be deformed.

Also, referring to FIG. 9(d), the degree of thermal deformation of the tissue may be controlled by controlling the temperature, flow rate, or the like, of the fluid. For example, the depth of the coagulated mucosa and muscle layer may be controlled by controlling the fluid injection conditions.

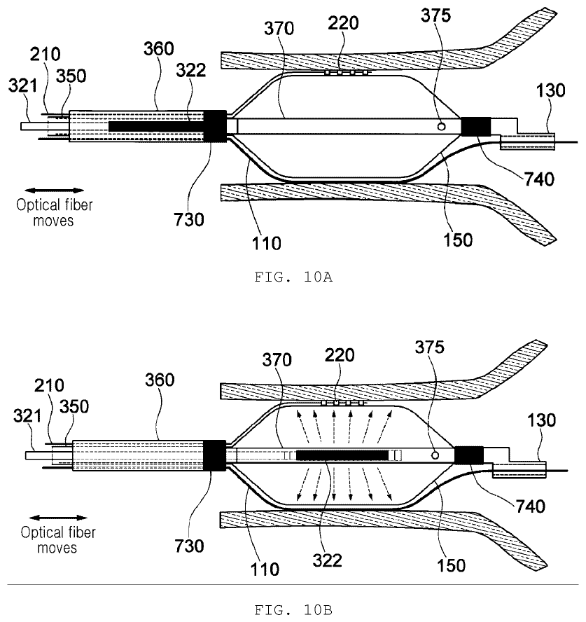

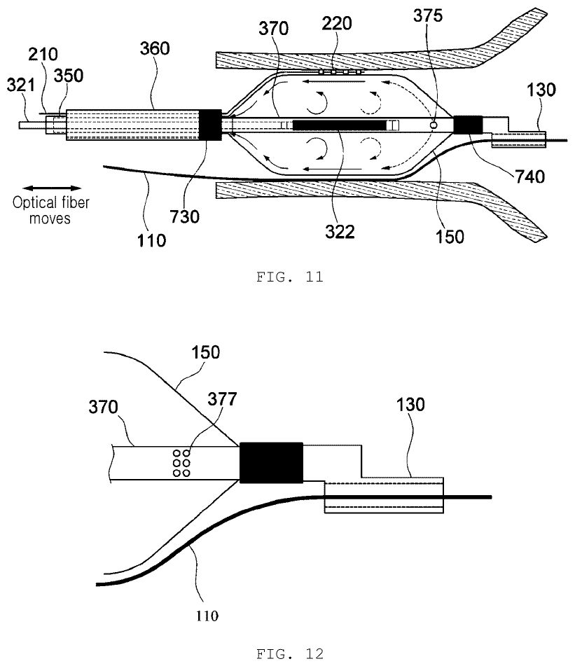

FIGS. 10A and 10B are diagrams s schematically illustrating a portion of optical fiber and a balloon portion of a phototherapy apparatus according to the other embodiment of the present disclosure. FIG. 11 is a diagram schematically illustrating movement of fluid in a balloon of a phototherapy apparatus according to an embodiment of the present disclosure.

The phototherapy apparatus illustrated in FIGS. 10a and 10b may be substantially similar to the phototherapy apparatus illustrated in FIGS. 1 to 9 other than the shape of the balloon portion 100, and thus, overlapping descriptions will not be provided.

Referring to FIGS. 10a and 10b, in an embodiment of the present disclosure, the balloon portion 100 may further include a third tube 370 into which the optical fiber 320 is inserted. In an embodiment, the third tube 370 may be disposed to be connected between one end of the first tube 350 and one end of the tip portion 130 and to accommodate the optical fiber 320 according to translational movement of the optical fiber 320. Also, the guide portion may not be provided in the balloon portion 100 according to the embodiment.

Referring to FIG. 10b, in an embodiment, when a light beam is irradiated for treatment, or the like, the light scattering portion 322 may be positioned in the third tube 370. Accordingly, the third tube 370 may be formed of a transparent material to not interfere with the light irradiation. For example, the third tube 370 may be formed of a material similar to that of the first tube 350. In an example, the third tube 370 may include at least one of Pebax, polyurethane, silicone, rubber, or PEEK (polyetheretherketone), but an embodiment of the present disclosure is not limited thereto. Also, the third tube 30 may be integrated with the first tube 350, and in this case, no seam may be present between the third tube 30 and the first tube 350.

Referring to FIGS. 10a-11, in an embodiment, the third tube 370 may include a hole 375 penetrating the third tube 370 to inject fluid into the balloon 150. That is, the fluid injected along the first tube 350 may flow into the third tube 370, may pass through the hole 375, and may be injected into the balloon 150. The fluid injected into the balloon 150 may move along the arrow and may be discharged between an outer surface of the first tube 350 and an inner surface of the second tube 360. In this case, the hole 375 of the third tube 370 may be disposed closer to the tip portion 130 than the second tube 360, and in this case, the cooling effect of the fluid may be further improved.

In an embodiment, the hole 375 of the third tube 370 in a plan view may have a circular, elliptical, streamlined, polygonal, slit, or irregular shape. However, an embodiment thereof is not limited thereto, and the hole 375 may have various shapes.

FIG. 12 is a diagram schematically illustrating a hole of a third tube of a phototherapy apparatus according to an embodiment of the present disclosure. The phototherapy apparatus illustrated in FIG. 12 is substantially similar to the phototherapy apparatus illustrated in FIGS. 10a to 11 other than the shape of the third tube 370, and thus, overlapping descriptions will not be provided.

Referring to FIG. 12, in an embodiment, the hole 377 of the third tube 370 may include a plurality of holes 377 disposed in the circumferential direction or the axial direction of the third tube 370. The shape, number, and arrangement of the holes 377 are not limited to the examples in FIG. 12, and the plurality of holes 377 may be arranged regularly or irregularly. Also, in FIG. 12, the plurality of holes 377 have a circular shape, but an embodiment thereof is not limited thereto.

The description of the present disclosure described above is exemplary, and a person having ordinary skill in the technical field to which the present disclosure belongs will understand that the embodiments may be easily transformed into other specific forms without changing the technical idea or essential features of the present disclosure. Therefore, it should be understood that the embodiments described above are exemplary and not limited. For example, the components described as integrated may be implemented in a distributed manner, and similarly, components described as distributed may be implemented in a coupled manner.

While the embodiments have been illustrated and described above, it will be configured as apparent to those skilled in the art that modifications and variations could be made without departing from the scope of the present disclosure as defined by the appended claims.

### REFERENCE NUMERALS

100: Balloon Portion 110: Guide Wire
120: Guide Portion 121: Guide Rail
130: Tip Portion 150: Balloon
200: Sensor Portion 300: Light Irradiation Portion
310: Optical Fiber Moving Portion 320: Optical Fiber
321: Light Transfer Portion 322: Light Scattering Portion
330: Light Generator 350: First Tube
360: Second Tube 370: Third Tube
375, 377: Hole 400: Fluid Adjustment Portion
500: Controller

1. A phototherapy apparatus, comprising:
a light generator configured to generate a laser beam;
an optical fiber coupled to the light generator and including a light transfer portion configured to transfer the laser beam and a light scattering portion configured to output the laser beam in a radial direction;
a first tube into which the optical fiber is inserted, and configured to inject fluid into one end;
a second tube into which the first tube is inserted, and configured to discharge the fluid in a region with an outer surface of the first tube;
a guide portion configured to accommodate the optical fiber by forward movement of the optical fiber and disposed on the same axis as the first tube; and
a balloon configured to surround one ends of the first tube and the second tube and the guide portion and to expand and reduce according to injection and discharge of the fluid.

2. The phototherapy apparatus of claim 1, further comprising:
a guide rail disposed between the first tube and the guide portion, and configured to guide movement of the optical fiber from the first tube to the guide portion.

3. The phototherapy apparatus of claim 1, wherein the guide portion has a circular, elliptical, streamlined, polygonal, or irregular cross-sectional shape and has a diameter decreasing in a direction away from the first tube.

4. The phototherapy apparatus of claim 1, wherein one end of the first tube is disposed between one end of the second tube and the guide portion.

5. The phototherapy apparatus of claim 1, wherein the first tube and the guide portion are spaced apart from each other such that the fluid is discharged between an outer surface of the first tube and the second tube after the fluid is injected into the balloon from one end of the first tube.

6. The phototherapy apparatus of claim 1, further comprising:
a fluid adjustment portion configured to control a flow rate and a temperature of the fluid.

7. The phototherapy apparatus of claim 1, further comprising:
an optical fiber moving portion configured to control the optical fiber to perform translational movement in an axial direction of the optical fiber or rotational movement in a circumferential direction of the optical fiber; and
a motor connected to the other end of the optical fiber.

8. The phototherapy apparatus of claim 7, wherein the optical fiber moving portion is configured to move the optical fiber in a first mode in which the light scattering portion is positioned in the second tube, and in a second mode in which the light scattering portion is positioned between the second tube and the guide portion.

9. The phototherapy apparatus of claim 1, wherein a length in an axial direction of the light scattering portion is smaller than a length in an axial direction of the balloon.

10. The phototherapy apparatus of claim 1, wherein at least one of the first tube or the balloon is transparent such that the laser beam passes therethrough.

11. The phototherapy apparatus of claim 1, further comprising:
a sensor array disposed on a surface of the balloon and configured to measure at least one of temperature, tissue deformation, pH, and mucosal impedance.

12. The phototherapy apparatus of claim 1, further comprising:
at least one of a first radio marker disposed on one side of the light scattering portion or a second radio marker disposed on one side of the balloon.

13. The phototherapy apparatus of claim 1, further comprising:
a tip portion disposed on a front end of the balloon; and
a guide wire configured to guide movement of the balloon,
wherein the guide wire is configured to penetrate the tip portion, to extend along one side of the balloon, and to be inserted into one end of the second tube.

14. A phototherapy apparatus, comprising:
a light generator configured to generate a laser beam;
an optical fiber coupled to the light generator and including a light transfer portion configured to transfer the laser beam and a light scattering portion configured to output the laser beam in a radial direction;
a first tube into which the optical fiber is inserted, and configured to inject fluid;
a second tube into which the first tube is inserted, and configured to discharge the fluid in a region with an outer surface of the first tube;
a third tube connected to one end of the first tube and configured to accommodate the optical fiber by forward movement of the optical fiber; and
a balloon configured to surround one ends of the first tube and the second tube and the third tube and to expand and reduce according to injection and discharge of the fluid,
wherein the third tube includes a hole to inject the fluid into the balloon.

15. The phototherapy apparatus of claim 14, wherein the third tube is transparent such that the laser beam passes therethrough.

16. The phototherapy apparatus of claim 14, wherein the fluid is injected into the balloon through the hole in the third tube and is discharged between an outer surface of the first tube and the second tube.

17. The phototherapy apparatus of claim 14, wherein the first tube and the third tube are configured as an integrated tube.

18. The phototherapy apparatus of claim 14, wherein a hole in the third tube is positioned closer to one side opposite to the first tube among both sides of the balloon.

19. The phototherapy apparatus of claim 14, wherein, on a plan view, the hole has a circular, elliptical, streamlined, slit, polygonal, or irregular shape.

20. The phototherapy apparatus of claim 14, wherein the hole includes a plurality of holes disposed in a circumferential direction or an axial direction of the third tube.
